(19) United States
(12) Patent Application Publication
(10) Pub. No.: US 2012/0294760 A1
(43) Pub. Date: Nov. 22, 2012

(54) METHOD AND APPARATUS FOR THE STERILIZATION OF PACKAGING MEANS

(76) Inventors: Heinz Humele, Thalmassing (DE); Andreas Kraus, Regensburg (DE); Roland Laumer, Regensburg (DE); Cornelia Folz, Berlin (DE); Jochen Forsthoevel, Regensburg (DE); Patrick Engelhard, Elsendorf (DE); Andreas Sonnauer, Woerth (DE)

(21) Appl. No.: 13/422,999

(22) Filed: Mar. 16, 2012

(30) Foreign Application Priority Data
Mar. 28, 2011 (DE) 10 2011 015 344.6

(51) Int. Cl. A61L 2/08; B01J 19/12

(52) U.S. Cl. 422/22; 422/186.05

(57) ABSTRACT

A method of sterilizing packaging means (10) and, in particular, plastics material pre-forms (10), wherein the packaging means (10) are conveyed with a conveying device (2) and wherein at least one area of the packaging means (10) is acted upon with a flowable medium by a stressing device (4). According to the invention the medium applied to the area is subjected to irradiation with electromagnetic radiation in order to produce singlet oxygen, wherein this singlet oxygen is used to sterilize the packaging means (10).

The present invention relates to a method and an apparatus for the sterilization of packaging means. It has long been known in the field of the drinks production industry for packaging means, such as for example glass or plastics material containers, packaging films, container closures and the like, to be sterilized before they are filled with a beverage for example. To this end the most widely varying sterilization methods are known from the prior art. In this way, it is known for example for the respective packaging means to be acted upon with peracetic acid or H₂O₂ for sterilization purposes. In addition, it is known for the packaging means to be acted upon with ultraviolet light or X-rays and the like. In the case of so-called wet disinfection in which peracetic acid is used or in the case of dry sterilization with the use of H₂O₂, there is the drawback that these involve aggressive media which are relatively difficult to handle and which therefore require complicated machine technology. In addition, the energy expenditure is relatively high and the drinking water circuit is contaminated with chemicals.

A method and an apparatus for irradiation by means of electron beams is known from DE 60 2004 011 056 T2. In this case the container is subjected to electron radiation and ozone is produced inside the container by the conversion of atmospheric oxygen. This ozone is kept in the closed container for a sufficient duration to sterilize the latter.

A method and an apparatus for the sterilization of containers for foodstuffs are likewise known from DE 697 15 399 T2. In this case the interior space of the container is irradiated with ultraviolet light shortly after the application of a solution containing hydrogen peroxide (H₂O₂).

DE 697 35 141 T2 likewise describes a method of sterilizing a container, in which the container is subjected to electron irradiation and ozone is produced inside the container by the conversion of atmospheric oxygen.

These named methods, which employ electron radiation or UV radiation, in turn have the drawback, however, that the aforesaid radiation has to be screened off since it can also be harmful to the user. These corresponding screening mechanisms are also relatively complicated in this case.

The object of the present invention is therefore to provide a method and an apparatus for the sterilization of packaging means, which does without aggressive substances on the one hand and also the use of radiation (for example ionizing) which is harmful to humans on the other hand.

These objects are attained according to the invention by the subjects of the independent claims. Advantageous embodiments and further developments form the subject matter of the sub-claims.

In the case of a method of sterilizing packaging means and, in particular, plastics material pre-forms, the packaging means are conveyed with a conveying device and at least one area of the packaging means is acted upon with a flowable medium by a stressing device. It is preferable for the flowable medium to be a photosensitizer present in solution (preferably in water).

According to the invention the medium applied to the area is subjected to irradiation with electromagnetic radiation in order to activate or to produce singlet oxygen, this singlet oxygen being used to sterilize the packaging means. In this case the flowable medium can contain oxygen, but it is preferable for the oxygen from the environment to be used. It is advantageous for the flowable medium to be a liquid.

As compared with the prior art, therefore, an alternative procedure is proposed in which so-called singlet oxygen is produced during the actual sterilization process. It is preferable for a photodynamic process to be initiated by the stressing with the electromagnetic radiation. An important mechanism in photodynamics is the absorption of light in dye molecules, which are also referred to as photosensitizers, and the possible processes which take place as a result. In this way, the absorbed light can be converted into heat in this photosensitizer or can be discharged again in the form of fluorescent light. For the third process, which is crucial for disinfection and sterilization respectively, part of the absorbed light energy can be transmitted to the surrounding oxygen, as a result of which an extremely reactive oxygen species is formed. The singlet oxygen generated in this way can subsequently damage irreparably and thus destroy the cell walls of bacteria and other micro-organisms. Which of the three named processes dominates can be controlled or influenced by the appropriate choice of the photosensitizer and the light dosimetry.

It is proposed to use this method known per se from the prior art for the sterilization of packaging means and, in particular, for the sterilization of plastics material pre-forms or the closures thereof and of machines and machine parts. An advantage of this method, in which use can also be made of dyes and also vitamins inter alia, is that photosensitizers remain after the application on the article to be sterilized. It would also be possible, however, for the photosensitizers to be subsequently removed, for example by a flushing process with sterile water or sterile air. In this way, it is advantageous for the flowable medium to be a so-called photosensitizer. An advantage of the method is that photosensitizers of this type are compatible with foodstuffs as a rule and vitamins for example are possible as the photosensitizers. In this way, it is also possible for flushing processes to be reduced or even eliminated. It is further possible for the above-mentioned activation of the photodynamic processes to take place only shortly before the processing of the item to be sterilized or during or after the processing respectively. If the photosensitizer is photostable, i.e. does not break down after the irradiation, the effects named above can be initiated once again.

Finally, by means of the procedure according to the invention the machine technology can be simplified and the use of chemicals can possibly be eliminated. In this way for example, the sterilization process can be initiated by the irradiation only immediately before an actual filling process of containers, as a result of which it is possible to reduce a sterile zone which is complicated in terms of both the method and the machinery. A further advantageous use of a photostable dye solution is the repeatable disinfection of machines or machine parts respectively. This is necessary, in particular, for the maintenance of the sterile zone.

The stressing of the packaging means is, in particular, a wetting of the respective surface areas, but an immersion of the packaging means into a bath of the flowable medium is also possible. The immersion bath treatment can be optionally assisted by the use of ultrasound. In addition, it is also possible for the aforesaid areas to be sprayed with the flowable medium. It is preferable to wet the entire internal surface of a packaging means to be sterilized or to distribute the flowable medium on the entire internal surface. It is advantageous for this wetting to be maintained during the entire following process and, in particular, also during the irradiation. It is advantageous for the wetting also to remain substantially constant during the entire process. For this purpose it is preferable for the flowable medium to be modified in such a way that it remains adhering in an improved manner to the faces of the packaging means or to the micro-organisms respectively. It may therefore be advantageous for the flowability and wetting capacity of the flowable medium to be adapted or altered.

It is advantageous for the aforesaid areas of the packaging means to be an inner wall of the same at least locally. It is advantageous for those areas of the packaging means or the plastics material pre-form—which in a subsequent closed state of the packaging means come into contact with the substance present in this packaging means, in particular the beverage present in this packaging means—to be acted upon with the flowable medium.

In the case of a further advantageous method the packaging means is acted upon with the flowable medium during the conveying and/or the singlet oxygen is produced or activated during the conveying of the packaging means.

It is advantageous in this case first for the aforesaid area of the packaging means to be acted upon with the flowable medium and then, only after a specified time (period of action), for the singlet oxygen to be activated by the irradiation (in particular electromagnetic irradiation and, in a particularly preferred manner, light in the visible wavelength range). In the case of a further advantageous method the medium also remains on the area of the packaging means for a pre-set period after the activation or irradiation, in order to achieve an efficient destruction of bacteria and germs in this way. It is preferable for a reduction rate of the micro-organisms in the range of 5 log levels (i.e. by the factor 10,000) to be achieved, this applying in particular to those areas of the packaging means which subsequently come into contact with the product, such as for example a beverage.

Within the scope of the technical implementation it would be possible for the flowable medium, i.e. a photosensitizer, to be sprayed into the packaging means, such as for example a bottle, the plastics material pre-form, a jar or even other hollow bodies which are, in particular, rotationally symmetrical and pressure-resistant or not pressure-resistant. The principle of rinsers known from the prior art can be used with this procedure. The dye (the photosensitizer) can be sprayed into the container by way of a single-duct or multiple-duct system and can be distributed. In this case it would be possible for the container to be held inverted or suspended. The article wetted on the inside and/or the outside or the packaging means is then irradiated with radiation, and in particular with light of a specified wavelength, and is thus sterilized. After that it is possible for one more flushing procedure to be carried out. The irradiation device in this case can also be a laser-based irradiation device. In this case lasers with a fixed or even tunable wavelength can be used. In addition, use can also be made of flash lamps, high-energy lamps, high-pressure lamps, LEDs, combinations thereof and the like.

As above, the flowable medium advantageously contains a dye. In particular, the medium is a dye or a dye solution. In addition, however, it would also be possible for the flowable medium to be applied to packaging means such as closures, sealing means, bulk materials, granulates or films, by means of an immersion bath and then for the packaging means acted upon in this way to be irradiated.

It is preferable for the flowable medium to be a substance, and in particular a dye, which is capable of producing the photodynamic effect. It is preferable for the dye to contain luminescent or reflecting particles which project the light required for the activation or excitation even into areas of difficult access having undercut edges for example. The reflecting particles ensure that the layer of dye is reflected in a diffuse manner. If a beam of light now strikes it, it is reflected in all free spatial directions. This makes it possible in an advantageous manner to reach even covered sides of a machine or the internal shouldered face of a bottle. In the luminescence method (fluorescence or phosphorescence) molecules or particles which are luminescent are added to the dye. If these are illuminated, they themselves start to illuminate in a non-directed manner and thus guide the light into covered areas. It is preferable for the excitation wavelength of the luminescent molecules or particles to be less than in the case of the dyes, in order to meet the excitation wavelength of the dyes in the emission. In addition, in the case of phosphorescent molecules or dyes it is also possible for the duration of the illumination to be increased (after-illumination) by a brief excitation and thus for even longer photodynamic processes to be carried out. In this way, even angled structures can be reached in an economical manner without undue outlay in terms of illumination.

In the case of a further advantageous method the stressing of the packaging means takes place before and/or during and/or immediately after heating of the packaging means. In particular, if the packaging means is a plastics material pre-form, it is known from the prior art for these to be shaped by a blow moulding machine or stretch blow moulding machine into plastics material containers. Before this shaping process the plastics material pre-forms are usually heated with a heating device, such as for example an infrared or microwave oven. In this case it is proposed that the stressing of the packaging means with the flowable medium and optionally also the activation of the singlet oxygen should take place at least in part at the same time for example before or during this heating procedure. The dwell period in the oven can be utilized for the sterilization of the pre-forms previously acted upon with the medium. The radiation in the heating process is used for the activation of the sterilization process and for the maintenance thereof until the sterile filling. It would also be possible for the flowable medium to be heated in a purposeful manner by the heating device (before or after the irradiation) for evaporation purposes for example. In addition, it would also be possible for the flowable medium to be discharged between a heating device and a stretch blow moulding machine onto the (inner) wall of the plastics material pre-form.

In the case of a further advantageous method the flowable medium is supplied to the packaging means by way of a supply line and/or at least one nozzle. Similar methods, by which for example H₂O₂ or peracetic acid or the like is applied to the inner wall of plastics material pre-forms, are known in this case from the internal prior art of the Applicants.

Furthermore, it would also be possible, in particular when used for bulk materials or granulates, for the latter to be introduced into a container such as a rotary drum and to be acted upon with the flowable medium there. Further packaging means, such as closures or sealing means, can be conveyed for example with a conveying system by way of worms or belts in a closed system, in which case the source of activation radiation is shut off from the latter.

Furthermore, it is also possible for the packaging means, in particular plastics material pre-forms, to be conveyed and to be taken past nozzle blocks. In particular, an external sterilization of the plastics material pre-forms or the packaging means is made possible by this procedure.

It is advantageous for the flowable medium to be a positively or a negatively charged liquid. In this way, an easier adhesion to micro-organisms can be achieved. In general, it is advantageous for the flowable medium to be adapted to the micro-organisms to be destroyed. The micro-organisms which are to be destroyed with the method described here are for the most part grampositive, i.e. negatively charged. For these micro-organisms it is preferable for a positive dye to be used. Some micro-organisms such as for example aspergillus niger can also, however, be positively charged, so that a negatively charged liquid or a negatively charged dye is appropriate in this case.

In order to increase the efficiency and/or to ensure the process ratios, oxygen can advantageously be added to the process in a purposeful manner and under controlled conditions.

Furthermore, it is expedient to use an illuminant with a large wavelength range for combining the photodynamic sterilization and the sterilization by UV light.

If the photosensitizers remain on or in the packaging means on account of their compatibility with foodstuffs (non-toxic effect) and if they are photostable, i.e. still capable of being activated in the process, it is possibly advantageous for this photostability to be destroyed in a purposeful manner after the sterilization by irradiation and/or tempering, so that an undesired reaction will no longer occur subsequently for example in the product.

In the case of a further advantageous method the stressing and the sterilization of the packaging means follow in a continuous process, in particular on a rotary machine. This means that the packaging means are guided at least locally along a circular path and during this conveying on this circular path they are also acted upon with the flowable medium and, in a particularly preferred manner, also with the electromagnetic radiation.

It is advantageous for the stressing device to move jointly, at least locally, with the packaging means to be sterilized, such as a plastics material pre-form. In addition, it would be possible for the irradiation device to move jointly, at least locally, with the packaging means. In this way, it would be possible for example for a plurality of stressing devices and preferably also irradiation devices to be arranged on a carrier wheel and also holding devices for holding the plastics material pre-forms to be arranged on the same carrier wheel. In this case a control device can be further provided which has the effect that first of all the plastics material pre-form is acted upon with the flowable medium at each sterilization station of this type and after that the irradiation of the areas acted upon starts after a pre-set period of time.

In the case of a further advantageous method at least one parameter of the flowable medium is monitored, in particular before it is applied to the packaging means. In this way, it is possible for example for a temperature of the flowable medium to be determined. In addition, it would be possible for measurements of the through-flow and concentration to be carried out (in particular in-line). Measurements of the filling height can also be carried out (for example whilst using an immersion bath).

In addition, it would also be possible for the consumption of the flowable medium to be determined, in particular in order to allow conclusions to be drawn on adequately wetted packaging articles in this way. It would also be possible for the irradiation to determine characteristic data, such as for example radiation energy or radiation power. In this way, an irradiation dose can be determined.

The present invention further relates to an apparatus for the sterilization of packaging means and, in particular, of plastics material pre-forms. This apparatus has in this case a conveying device which conveys the packaging means along a pre-set conveying path as well as a stressing device which acts upon at least one area of the packaging means with a flowable medium.

According to the invention the apparatus additionally has an irradiation device which subjects the area of the packaging means acted upon with the flowable medium to an electromagnetic radiation in order to produce singlet oxygen.

It is advantageous in this case for the irradiation device to have at least one and preferably a plurality of light-emitting diodes. It would also be possible, however, for a flash lamp or a high-energy lamp to be used as the light source (in addition or as an alternative).

If the packaging means is a plastics material pre-form, it would be possible in this case for the radiation source to be arranged outside the plastics material pre-form and to illuminate the latter from the outside, i.e. through its wall, in order to activate the singlet oxygen. It would also be possible, however, for the light source to be introduced into the interior of the plastics material pre-form. In addition, it would also be possible for the irradiation device to irradiate the plastics material pre-forms or the inner wall thereof from the aperture. Reflector elements could also be introduced into the plastics material pre-form and could be illuminated from the outside.

It is advantageous for the conveying device to convey the plastics material pre-form along a circular path. In addition or as an alternative, it would also be possible for plastics material pre-forms also to be moved or conveyed along the longitudinal direction thereof. In this way, it would be possible for the plastics material pre-forms to be conveyed through a heating device, such as a microwave heating device, and also to be sterilized during this procedure. In addition, it would also be possible, however, for the plastics material pre-forms to be conveyed along a path which is straight at least locally.

In the case of a further advantageous embodiment the conveying device has a holding means for holding the packaging means, and the stressing device and/or the irradiation device is or are preferably also incorporated into this holding means. The holding means in this case is preferably a holding means which contacts the plastics material pre-form on an inner wall. In this way, the holding means can be for example a mandrel which engages in an aperture of the plastics material pre-form. The aforesaid stressing devices, for example in the form of nozzles or even openings, can be arranged in this case in the region of this mandrel and preferably in that region which projects into the plastics material pre-form. In addition, the irradiation device, which can be light-emitting diodes for example, can be incorporated in this holding means.

In this way, the holding means preferably engages in an aperture of the packaging means. It is preferable for the apparatus to have a plurality of holding means of this type and in a particularly preferred manner the latter are arranged equidistantly from one another. It is advantageous for the face on which the holding means contacts the plastics material pre-form to be minimized. In the case of a further advantageous embodiment openings are also incorporated into the holding means in order to act upon the plastics material pre-form with the flowable medium in the contacted areas as well.

In the case of a further advantageous embodiment the apparatus has a heating device in order to heat the packaging means, and in particular the plastics material pre-form. In a particularly preferred manner this heating device can be a microwave-based heating device. It is advantageous for the aforesaid apparatus to have arranged downstream of it a stretch blow moulding machine in the conveying direction of the packaging means.

In the case of a further advantageous embodiment it would also be possible for the plastics material pre-form to be rotated with respect to its longitudinal axis and relative to the stressing device. In this way it is possible for the inner wall of the plastics material pre-form to be irradiated over its entire periphery, even if the stressing device delivers the flowable medium only in one (in particular oblique) direction. It would also be possible, however, for a plurality of radiation devices which illuminate the plastics material pre-form from a multiplicity of directions to be used. In this way, it is possible to avoid shadow casting which in certain regions of the plastics material pre-form prevents the production of singlet oxygen. In order to optimize the yield of the irradiation, use can advantageously be made of reflectors, for example mirrored surfaces.

Furthermore, it would also be possible for the plastics material pre-form to be conveyed obliquely along a circular path. In this way it is possible for the flowable medium to pass downwards more rapidly inside the plastics material pre-form as a result of centrifugal force and thus for the entire internal surface of the plastics material pre-form to be wetted with the flowable medium. Furthermore, in the case of this embodiment it would also be possible for an irradiation device to be provided which rotates with respect to the plastics material pre-form in order to be irradiated in this way from all sides in its peripheral direction. In this way, for example, a light strip which is arranged outside the plastics material pre-form and which rotates with respect to the plastics material pre-form could also be provided. It would also be possible, however, for the light source to be made annular or in the form of annular segments and to move the plastics material pre-form through this light source which is made annular or in the form of annular segments.

In the case of a further advantageous embodiment the apparatus has a clean room. In this way, it is possible for the packaging means such as plastics material pre-forms to be conveyed through a clean room or sterile room of this type during the stressing with the flowable medium and preferably also during the irradiation. The sterility of the plastics material pre-form can be improved in this way. It would also be possible, however, for a clean room of this type only to be attached directly to the apparatus described here, so that the sterilized plastics material pre-forms pass directly into the aforesaid clean room. It is preferable for the irradiation device to supply light in the visible wavelength range.

Since a specific irradiation or light dose is necessary for the process, preferably at least 10 J/cm2, this process could lead to the plastics material pre-form being heated as a result of absorption. This heating can also be utilized as energy for the heating procedure of the plastics material pre-form. It would also be possible, however, for the process described here to take place after the heating of the plastics material pre-forms, so that for example a tempered dye solution is introduced into the plastics material pre-form after a heating process, and then to be left to be absorbed, then for the plastics material pre-form to be irradiated and finally to be shaped in a stretch blow moulding process to form a plastics material container.

It is advantageous for the apparatus according to the invention to be arranged in an aseptic block, in particular in combination with an aseptic stretch blow moulding machine arranged adjoining.

The advantages of in particular disinfecting the plastics material pre-form are that as compared with the finished container the plastics material pre-form has a small surface which has to be sterilized. In this way, the outlay in spraying on the dye solution is also less. Furthermore, as compared with the finished blow moulded container the plastics material pre-forms have a simpler geometry and, in particular, have few or no undercuttings. This is advantageous in particular during the application of the dye (for example by spraying) but also during the application with radiation.

Furthermore, the manipulation of plastics material pre-forms is comparatively simple, in which case a smaller distribution is also possible, so that as a whole a more compact machine technology can be used.

It is pointed out, however, that the present invention can also be used for other containers and other plastics material containers, such as plastic bottles.

Further advantages and embodiments may be seen in the accompanying drawings. In the drawings

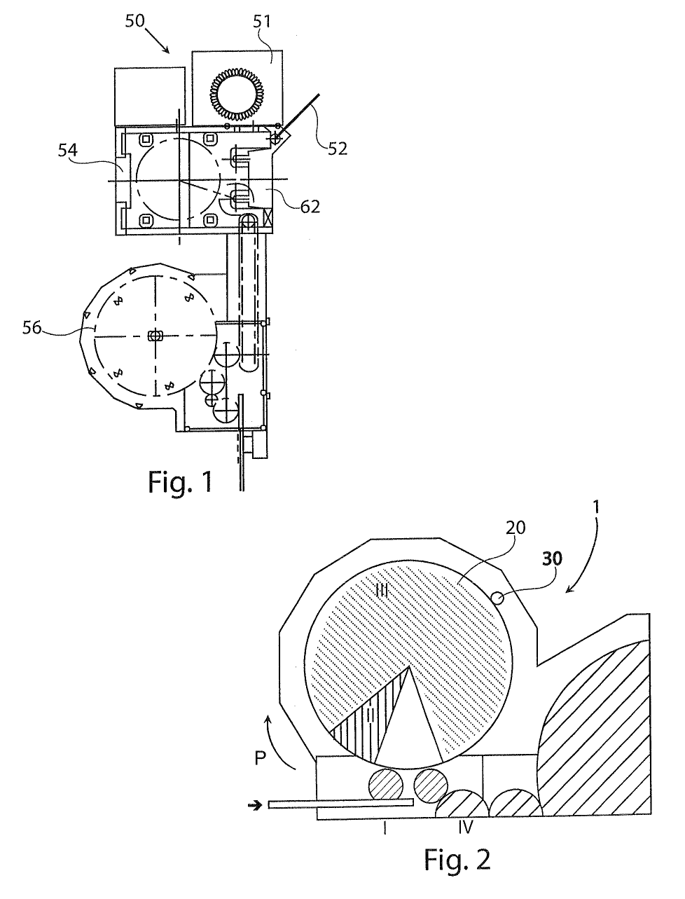

FIG. 1 is a diagrammatic view of a plant for the production of containers;

FIG. 2 is a detailed view to explain the time sequences of a sterilization procedure;

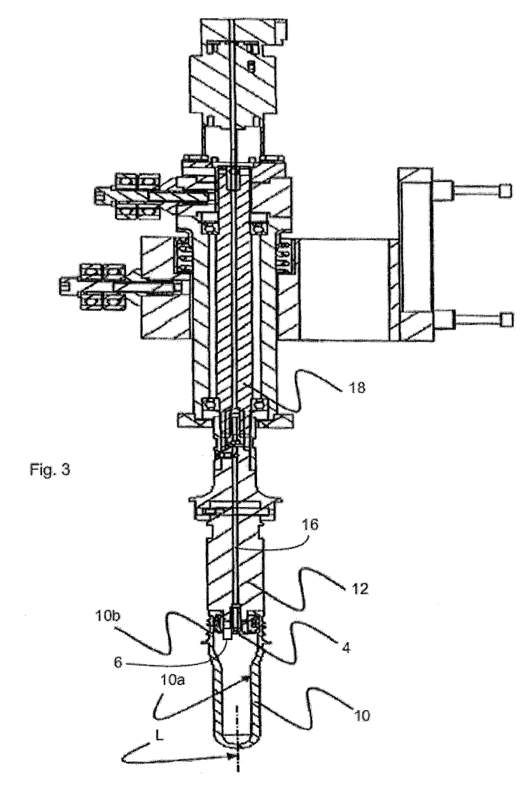

FIG. 3 shows a device for the sterilization of plastics material containers;

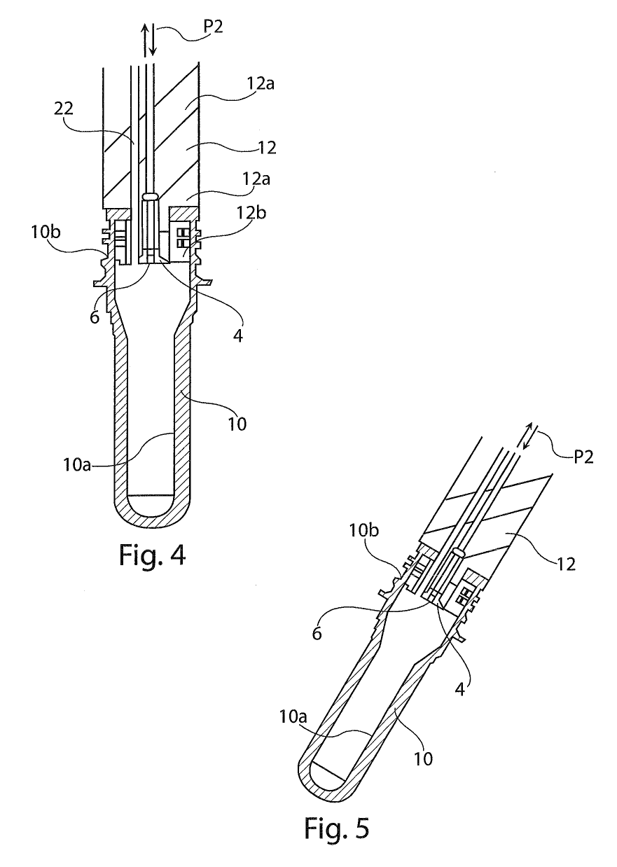

FIG. 4 is a detailed view of the apparatus shown in FIG. 3;

FIG. 5 shows a further embodiment of an apparatus for the sterilization of containers;

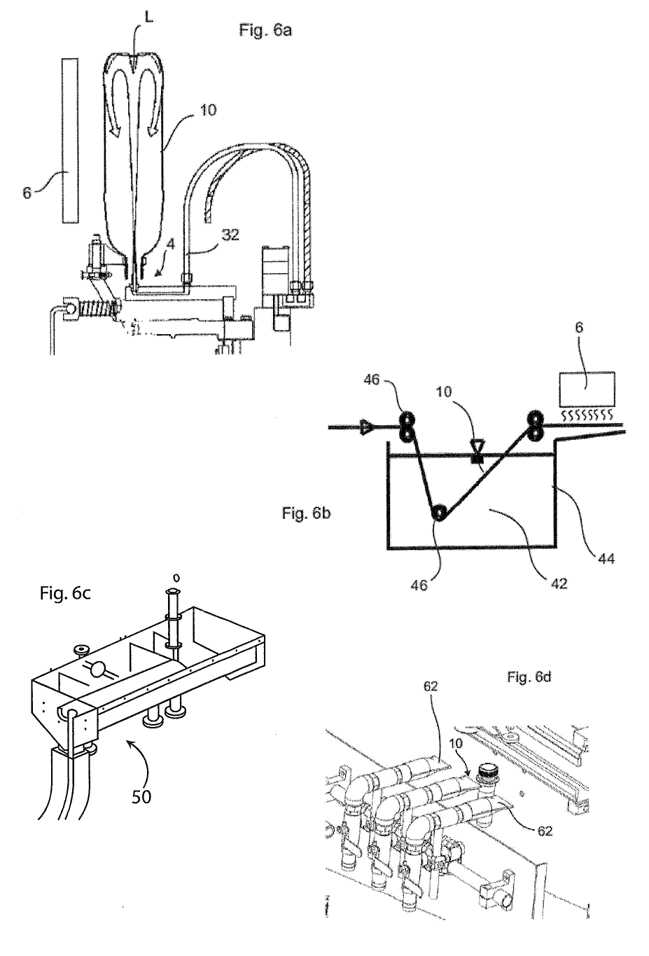

FIGS. 6a-6d show four embodiments of further apparatus for the sterilization of containers, and

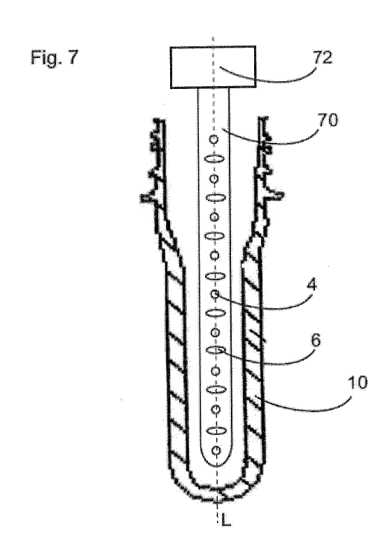

FIG. 7 shows a further embodiment of a sterilization device according to the invention.

FIG. 1 shows a plant 50 for the handling of containers. In this case the reference number 52 relates to a supply device which transfers the plastics material pre-forms to a heating device or a oven 51. This heating device 51 can in this case be a microwave oven, inside which the containers or plastics material pre-forms respectively are conveyed along a circular path and are heated in resonators. It would also be possible, however, for the apparatus to have IR heating elements which can also be designed in the form of heating cavities for the individual heating of the plastics material pre-forms. This heating device 51 is adjoined by a stretch blow moulding machine 54, inside which the plastics material pre-forms are heated to form plastics material containers. After that, the containers are filled by a filling machine 56 and are conveyed away.

As mentioned above, the apparatus 1 according to the invention is preferably used for the sterilization of plastics material pre-forms. In this way it would be possible for the plastics material pre-forms to be sterilized during the conveying in the supply device 52, but it is preferable for the plastics material pre-forms to be sterilized during the heating in the heating device 51. In addition, it would also be possible for the plastics material pre-forms to be sterilized after leaving the heating device 51 and during the conveying to the stretch blow moulding machine 54. Sterilization of the finished containers after leaving the stretch blow moulding machine 54 would also be possible.

Furthermore, it would be possible for the plastics material pre-forms to be conveyed in a sterile room or clean room 62 and to be both heated and shaped to form plastics material containers inside this clean room. In this case, as mentioned above, the sterilization can be carried out in this sterile room 62 but also for example immediately before entering this sterile room 62. It would also be possible, however, for the sterilization device 1 to be designed in the form of a separate module which can also be added for example in the case of existing plants. Furthermore, the plant 50 can have further sterilization units, for example for the sterilization of the external surfaces of the plastics material pre-forms and the like.

FIG. 2 shows diagrammatically the course of a sterilization method according to the invention. The plastics material pre-forms can run into the sterilization plant 1 along a supply portion I and can be acted upon and then irradiated with the flowable medium in the region of portion II. The reference letter P in this case designates the conveying path of the plastics material pre-forms. In portion III the now activated flowable medium can act upon the plastics material pre-forms and in portion IV the treated plastics material pre-forms can move out of the machine again. In this case it would also be possible, as mentioned above, for heating of the plastics material pre-forms to take place, in particular in sections II and III. It would be possible in this case for a plurality of sterilization stations 30 (only one shown diagrammatically) to be arranged on a rotatable carrier 20.

FIG. 3 shows a handling unit for handling the plastics material pre-forms. A plastics material pre-form 10, which is arranged on a holding mandrel designated 12 in its entirety, is evident here.

The reference number 4 relates to a stressing device which acts upon the interior of the plastics material pre-form 10, and in particular the inner wall 10a thereof, with a flowable medium. The reference number 10b designates an aperture of the plastics material pre-form.

Furthermore, it is possible for the holding means or the holding mandrel 12 to have irradiation devices 6 which act upon the inner wall 10a of the plastics material pre-form 10 with light in a purposeful manner in order to activate the flowable medium and to produce the singlet oxygen respectively in this way.

The reference number 16 designates a supply line through which the flowable medium can be conveyed to the stressing device 4. This supply device is situated in the interior of the holding mandrel 12 here.

The reference number 18 designates a movement unit which is used for moving the plastics material pre-forms. First of all this movement unit 18 permits a raising and lowering movement in order to introduce the plastics material pre-form 10 into the resonator of a heating device from above along its longitudinal axis L for example and also to withdraw it again.

Furthermore, the movement unit 18 can also be used in order to turn the plastics material pre-form 10 with respect to its longitudinal axis L, in particular while it is being heated. In this case the stressing device 4 carries the flowable medium substantially on the entire internal surface of the plastics material pre-form. After the irradiation with the irradiation device the flowable medium can carry out its sterilization action and can sterilize the inner wall 10a of the plastics material pre-form.

FIG. 4 is an enlarged illustration of the apparatus shown in FIG. 3. Here too the holding means 12 on which the plastics material pre-form 10 is held by way of its thread 10b may be seen. As a result of its abutment against the inner face this internal gripping means produces a “shadow” or a face which is possibly not reached by the irradiation or the stressing.

It should thus be noted that these faces are as small as possible or that there are no shadows. Alternatively, the pre-form or the container can also be held on the outside and conveyed. It would also be possible for the internal gripping means to be made transparent so that at least the radiation passes through it. The stressing could be carried out before this (transparent) internal gripping means takes up the plastics material pre-form.

Furthermore, a removal duct 22 by way of which the removal of a medium—in particular a gaseous medium—is possible may be seen here. If the flowable medium penetrates into the plastics material pre-form 10 by way of the stressing device 4, in this way it displaces, in particular, air or sterile air inside the plastics material pre-form, which air can then be removed by way of the removal duct 22. Furthermore, it would also be possible for the holding means 12 to be constructed in two parts, for example with a rotationally fixed region 12a and a region 12b which rotates with respect to the latter. In this way, it would be possible for the plastics material pre-form 10 to be rotated, while the stressing device 4 is held in a rotationally fixed manner. In this way, the inner wall 10a of the plastics material pre-form can be wetted with the flowable medium by a rotation of the plastics material pre-form also in the peripheral direction. Furthermore, it would also be possible for the stressing device 4 or the nozzle to be acted upon with the sterilization medium by way of supply and or removal lines in each case and/or for this sterilization medium to be drawn out of the plastics material pre-form 10 in order to develop its sterilizing action in particular on the internal surface 10a of the plastics material pre-form 10 by additional heating. FIG. 5 is a further illustration of an apparatus according to the invention. It is evident that in this case the plastics material pre-form 10 is set in an oblique manner. This embodiment is advantageous in particular when the plastics material pre-form is moved along a circular path. On the one hand a deformation of the heated plastics material pre-form can be better prevented in this way, and on the other hand this arrangement also permits a more advantageous wetting of the entire internal surface 10a of the plastics material pre-form.

In this case it would again be possible for the plastics material pre-form also to be rotated with respect to its longitudinal axis so that in this way a still more advantageous distribution of the sterilization medium on the inner wall of the plastics material pre-form is achieved.

FIGS. 6a to 6d show four further possible procedures in the sterilization of packaging means. In the case of the variant shown in FIG. 6a which is also used in particular for the sterilization of already finished containers 10, recourse can be had to the existing principle of flushers (rinsers). By way of a supply duct 32, in which case a single-duct or a multiple-duct system can also be provided, the dye (sensitizer) is sprayed and distributed into the plastics material container 10 held by the holding means 14. The distribution can be carried out in this case in the form of a dispersion or solution for example.

In this case the packaging means or here the container 10 can also be present inverted or suspended. The container wetted on the inside and/or the outside is then irradiated with light of specified wavelength by the irradiation device 6 and is thus sterilized. After that, a further flushing procedure can be carried out. In this case the irradiation device 6 can be arranged around the container over the entire periphery, but it would also be possible for the container 10 to rotate with respect to its longitudinal axis L.

The variant shown in FIG. 6b is suitable in particular for film-like packaging means 10. In this case the packaging means is conveyed through a container 44 in which the flowable medium is present. For this purpose a plurality of rollers 46 are provided about which the packaging means 10 extends.

After that, the packaging means is likewise irradiated with the irradiation device 6 which is arranged stationary in this case. This procedure could also be used for granulates or even closures.

In the case of the variant shown in FIG. 6c a conveying system 50 is provided which has a plurality of worms and/or guides in a closed system in order to convey the packaging means. The irradiation device (not shown) can also be designed to be shut off from the actual conveying system 50 here. The packaging means 10 here can be in particular container closures. These can be conveyed with their inner side towards the top for example and in this case can first be acted upon with the flowable medium and after that with the irradiation device.

FIG. 6d shows a further procedure for the irradiation of plastics material pre-forms. Here a plurality of nozzle blocks 62 are provided, by means of which the plastics material pre-form 10 is acted upon on its outer wall with the flowable medium. In addition, irradiation devices (not shown) which activate the flowable medium on the outer wall of the plastics material pre-form can also be provided here.

FIG. 7 shows a further embodiment of the sterilization of plastics material pre-focus 10. In this case a rod-like body 70 which is inserted into the interior of the plastics material pre-forms 10 is provided here. In this case it would be possible for the rod-like body to move along its longitudinal direction, but it would also be possible for the plastics material pre-form to be moved.

Here this rod-like body 70 has provided in it both the stressing devices 4 in the form of openings and the irradiation devices 6 which can be cast in the form of LEDs for example into this rod-like body 70 or can be incorporated in the latter in some other way (by screw fastening, snug fitting and the like). In this case it would be possible for the stressing devices 4 and the irradiation devices 6 to be arranged in each case around the rod-like body 70 over the entire periphery here, but it would also be possible for it to point only in one direction, and for the rod-like body 70 to rotate with respect to the longitudinal direction L of the plastics material pre-form 10.

It would also be possible for the rod-like body 70 to be held in a rotationally fixed manner and, conversely, for the plastics material pre-form to rotate. In this case the rod-like body could be produced from a plastics material for example, which would be advantageous in particular for applications in conjunction with microwave-based heating devices. Stressing devices 4 and optionally also irradiation devices 6 could also be provided at the lower end of the rod-like body.

In addition, in particular in the case of transparent plastics material pre-forms, it would also be possible for the stressing devices 4 again to be arranged outside the plastics material pre-form.

The reference number 72 designates in a roughly diagrammatic manner a drive for the rod-like body, which is used in this case for inserting the rod-like body into the plastics material pre-forms and which can optionally also carry out a rotational movement of the rod-like body 70.

A wetting of the plastics material pre-forms could also be carried out in such a way that they are first immersed completely in a bath with the flowable medium and are thus filled with the latter and are then emptied again, in particular by turning the plastics material pre-forms. In this case the flowable medium can act upon the inner wall of the plastics material pre-forms for a pre-set time and, after that, these plastics material pre-forms can be irradiated (from the inside or the outside) in order to produce singlet oxygen. The oxygen can be derived in this case from the ambient air, but it would also be possible for additional oxygen to be added. In this case it would also be possible for the plastics material pre-forms to be conveyed past irradiation devices and, preferably also in this case, to rotate about their longitudinal axis.

The Applicants reserve the right to claim all the features disclosed in the application documents as being essential to the invention, insofar as they are novel either individually or in combination as compared with the prior art.

### LIST OF REFERENCES

1 apparatus
4 stressing device
6 irradiation device
10 plastics material pre-form
10a inner wall
10b aperture of the plastics material pre-form, thread
12 holding mandrel
12a rotationally fixed region
12b rotating region
14 holding means
16 supply line
18 movement unit
20 rotatable carrier
22 removal line
30 sterilization station
32 supply duct
44 container
46 rollers
50 plant, conveying system
51 oven, heating device
52 supply device
54 stretch blow moulding machine
56 filling machine
62 clean room, sterile room, nozzle block
70 rod-like body
72 drive
L longitudinal axis
P conveying path
I, II, III, IV portion, region

1. A method of sterilizing packaging material and, in particular, plastics material pre-forms, wherein the packaging material is conveyed with a conveying device and wherein at least one area of the packaging material is acted upon with a flowable medium by a stressing device, wherein the medium applied to the area is subjected to irradiation with electromagnetic radiation in order to produce singlet oxygen, wherein this singlet oxygen is used to sterilize the packaging material.

2. A method according to claim 1, wherein the packaging material is acted upon with the flowable medium during the conveying and/or the singlet oxygen is produced during the conveying of the packaging material.

3. A method according to claim 1, wherein the flowable medium contains a dye.

4. A method according to claim 1, wherein the stressing of the packaging material takes place before and/or during and/or immediately after heating of the packaging material.

5. A method according to claim 1, wherein the flowable medium is supplied to the packaging material by way of a supply line and/or at least one nozzle.

6. A method according to claim 1, wherein the flowable medium is a positively or a negatively charged liquid.

7. A method according to claim 1, wherein the stressing and the sterilization of the packaging material follow in a continuous process, in particular on a rotary machine.

8. An apparatus for the sterilization of packaging material and, in particular, of plastics material pre-forms, with a conveying device which conveys the packaging material along a pre-set conveying path, with a stressing device which acts upon at least one area of the packaging material with a flowable medium, wherein the apparatus has an irradiation device which subjects the area of the packaging material acted upon with the flowable medium to an electromagnetic radiation in order to produce singlet oxygen.

9. The apparatus according to claim 8, wherein the conveying device has a holding device for holding the packaging material, and the stressing device and/or the irradiation device is or are preferably also incorporated in this holding device.

10. The apparatus according to claim 9, wherein the holding device engages in an aperture of the packaging material.

11. The apparatus according to claim 8, wherein the apparatus has a heating device in order to heat the packaging material.
